(19) United States
(12) Patent Application Publication
(10) Pub. No.: US 2004/0268434 A1
(43) Pub. Date: Dec. 30, 2004

(54) Plant transformation

(76) Inventors: Thomas Leustek, Union, NJ (US); Yuying Luo, Highland Park, NJ (US)

Correspondence Address: Patent Department, Choate, Hall & Stewart, 53 State Street, Exchange Place, Boston, MA, 02109, US

(21) Appl. No.: 10/805,135

(22) Filed: Mar. 19, 2004

Related U.S. Application Data
(63) Continuation of application No. PCT/US04/08268, filed on Mar. 18, 2004.
(60) Provisional application No. 60/455,482, filed on Mar. 18, 2003.

(51) Int. Cl. C12N 15/82; C12N 15/87

(52) U.S. Cl. 800/278; 800/294; 800/288

(57) ABSTRACT

The present invention provides improved methods and reagents for transforming plant cells, and particularly for Agrobacteriun tumefaciens-mediated transformation of Brassica juncea.

The present application is a continuation of, and claims priority to, PCT/US ______, filed Mar. 18, 2004, which claims priority to U.S. Provisional Application No. 60/455,482, filed Mar. 18, 2003, each incorporated herein by reference.

### BACKGROUND

Brassica juncea is a plant species in the Brassicaceae family that has great potential as a crop plant, a phytoremediation candidate, a delivery vehicle for micronutrients in the human diet, and as a factory for pharmaceutical proteins. A number of closely related and morphologically similar species are major commodities. For example, Brassica oleraceae is used commercially for production of Canola vegetable oil. Other species and varieties of Brassica are popular commercial vegetables, including cabbage, broccoli, cauliflower, and Brussels sprouts. Clearly, it would be valuable to be able to efficiently transform plants in the Brassicaceae family. Unfortunately, however, available transformation techniques often find only limited success when applied to Brassicaceae family members, and particularly when applied to B. juncea.

The most widely used method for introduction of new genes into plants is based on the natural DNA transfer capacity of Agrobacterium tumefaciens. The currently available methods for genetic transformation of B. juncea are based on the infection of cell cultures with A. tumefaciens. Since 1990, several publications have reported tissue culture methods for transformation of B. juncea (Mathews et al., Plant Sci. 72:245,1990; Chi et al., Plant Cell Rep. 9:195, 1990; Zhu et al., Plant Physiol. 119:73, 1999; and Prasad et al., Plant Sci. 159:233, 2000). All of these publications describe relatively low frequencies of transformation. The highest reported was able to coax 9% of initial explants to grow into transformed plants. All of the published procedures are also very laborious, requiring up to 1 year to transfer from in vitro cultivation into potting mix in an open environment.

### SUMMARY

The present invention provides a superior system for Agrobacterium-mediated gene transfer into Brassica species. The invention defines transformation parameters that together increase transformation efficiency to greater than 25%, preferably greater than 30%, more preferably greater than 35%, 40%, 45%, 50%, 55%, or even 60% and/or reduce the time required to generate transformed plants to not more than 3 months, preferably not more than 2 months, and optimally within about a month.

In certain preferred embodiments, the inventive system is applied to production of therapeutically and/or nutritionally significant proteins.

### BRIEF DESCRIPTION OF THE DRAWING

The invention is described herein with reference to several Figures of the drawing, in which:

FIG. 1 is a graph showing the effect of different re-culture conditions on shoot regeneration from cotyledon, hypocotyls and cotyledonary petiole of B. juncea after transformation by A. tumefaciens.

FIG. 2 is a graph showing the effect of activation of Agrobacterium on shoot regeneration from cotyledon, hypocotyls and cotyledonary petiole of B. juncea after transformation by A. tumefaciens.

FIG. 3 is a graph showing the effect on shoot regeneration from cotyledon, hypocotyls and cotyledonary petiole of B. juncea of wash treatment of explants after co-culture and transformation by A. tumefaciences.

FIG. 4 is a graph showing the effect of growth regulators on shoot regeneration from cotyledon, hypocotyls and cotyledonary petiole of B. juncea after transformation by A. tumefaciens.

FIG. 5 is a photograph showing the high efficiency transformation of transgenic Brassica juncea shoots growing in the presence of 3 mg/L phosphenothricin.

FIG. 6 is a graph showing the luciferase activity measured over a 24 hour period after exposure to heat shock.

### DEFINITIONS

Abbreviations

Ω, Omega translational enhancer of tobacco mosaic virus

2iP, 2-isopentenyladenine

35S, major promoter of the cauliflower mosaic virus

BAP, benzylaminopurine

GUS, β-glucuronidase

HGH, human growth hormone

IAA, indole-3-acetic acid

NAA, naphthalene acetic acid

NOS, nopaline synthase

PCR, polymerase chain reaction

TDZ, thidiazuron

Definitions

Administration of a compound or agent to a cell, tissue, or organism according to the present invention encompasses any and all modes of delivery. Where the administered entity has biological or therapeutic activity, such delivery should preferably be accomplished in a manner that preserves or protects at least some of the activity. Oral administration, for example, refers to delivery by way of the digestive tract, and is not limited to delivery by mouth (e.g., eating or drinking).

Expression of a gene refers to production of a gene product (e.g., primary transcript, mRNA, pre-protein, protein, higher order complex) and can involve one or more of the processes of transcription, splicing, RNA processing, translation, protein trafficking, post-translational modification, and/or oligomerization.

Expression cassette or expression vector refers to a nucleic acid molecule that includes one or more control elements sufficient to direct expression of a linked sequence.

A food or food product is any liquid or solid material appropriate for ingestion by humans or other animals. In some embodiments of the invention, edible plants or plant portions are transformed, and the transformed plant tissue is incorporated into a food or food product. In certain preferred embodiments, such a food or food product comprises raw plants or plant tissue. Alternatively, a plant-based food or food product may be prepared by any known method, including, for example, concentration or condensation of solid plant matter to form, for example, a pellet; production of a paste; drying, or lyophilization; cutting, mashing, or grinding the plant to various extents; or extraction of the liquid part of the plant to produce a soup, a syrup, or a juice. A processing step can also include cooking (e.g., steaming) the plant.

A gene, as that term is used herein, refers to an expressible portion of a nucleic acid. Typically, genes will be associated with regulatory sequences such as promoter sequences, 5′ or 3′ untranslated sequences, and termination sequences. In addition, introns and exons may also be included. The gene and regulatory sequences may be derived from the same natural source, or may be heterologous to one another. In certain preferred embodiments of the invention, a gene includes a coding sequence for a polypeptide or protein.

Heterologous, as applied to nucleic acid sequences herein, means of different origin. For example, if a host cell is transformed with a nucleic acid sequence that does not occur in the untransformed host cell in nature, then that nucleic acid sequence is said to be heterologous to the host cell. Furthermore, different elements (e.g., promoter, enhancer, coding sequence, terminator, etc) of a transforming nucleic acid may be heterologous to one another and/or to the transformed host. The term heterologous, as used herein, may also be applied to nucleic acids that are identical in sequence to a nucleic acid already present in a host cell, but that are now linked to different additional sequences and/or are present at a different copy number, etc.

A pharmaceutically active agent, as that term is used herein, is one that aids or contributes to the condition of a recipient in a positive manner when administered in a therapeutically effective amount. For example, a pharmaceutically active agent may have curative or palliative properties against a disease, and/or may be administered to ameliorate relieve, alleviate, reverse or lessen the severity of a disease or disorder. Alternatively or additionally, a pharmaceutically active agent may have prophylactic properties and/or may be used to prevent the onset of a disease or to lessen the severity of such disease or pathological condition when it does emerge. Pharmaceutically active agents may include an entire protein or polypeptide or instead may include only pharmaceutically active fragments thereof. The term also encompasses pharmaceutically active analogs of the agent. Furthermore, the term may also be used to refer to a single entity (e.g., small molecule, protein, or plant), or to a collection or complex of entities, for example that act cooperatively or synergistically to provide a therapeutic benefit.

A promoter, as used herein, is a DNA sequence that initiates transcription of an associated DNA sequence. A promoter region may also include elements that act as regulators of gene expression such as activators, enhancers, and/or repressors.

Regulatory elements refer to sequences involved in conferring the expression of a nucleotide sequence. Regulatory elements include 5′ regulatory sequences such as promoters that can be linked to the nucleotide sequence of interest, 3′ sequences such as 3′ regulatory sequences or termination signals. Regulatory elements also typically encompass sequences required for proper splicing, translation, modification, trafficking, and/or modification of expression products.

Substantially isolated is used in several contexts and typically refers to the at least partial purification of a protein, polypeptide, small molecule, nucleic acid, etc. away from unrelated or contaminating components (for example, plant structural and metabolic proteins). Methods for isolating and purifying proteins, polypeptides, small molecules, nucleic acids, etc., are well known in the art.

Transformation refers to introduction of a nucleic acid into a cell, particularly the stable integration of a DNA molecule into the genome of an organism of interest.

### DETAILED DESCRIPTION OF CERTAIN PREFERRED EMBODIMENTS

The present invention provides a system for improving transformation efficiency in a plant. For example, the invention provides improved methods of 1) providing tissue explants; 2) pre-culturing tissue explants; 3) activating bacteria for transformation; 4) co-culturing plant and bacteria cells; and/or 5) growing shoots from transformed cells; any or all of these improvements may be used individually or in combination, in accordance with the present invention.

Plants

Preferred plants for use in accordance with the present invention are members of the plant family Brassicaceae. Members of this family include, but are not limited to cabbages, mustards and radishes. The most preferred members of this family belong to the tribe Brassiceae. Members of this tribe include mustards of the genus Brassica and related species, described in more detail below.

Preferred embodiments of the present invention utilize crop and/or crop-related members of the above-identified family and tribe. The term “crop member” refers specifically to species of the family Brassicaceae which are commercially grown as sources for vegetables, oilseeds, forage, fodder and condiments. Examples of crop members of the family Brassicaceae include, but are not limited to, digenomic tetraploids such as B. juncea (mustard), B. carinata (Ethopian mustard), and B. napus (rapeseed); and monogenomic diploids such as B. oleracea (cole crops), B. nigra (black mustard), and B. campestris (turnip rape).

The term “crop-related member” refers specifically to species of the family Brassicaceae that have shown potential for use in echnologies such as phytoremediation (in which the plants are used to remove heavy metals from soil, water, mine tailings or other locations where the high metal content poses a threat to human heath) or to plants that show potential as indicators or sentinels of heavy metal contamination (e.g., so that the plants can serve to identify or map locations of heavy metal concentration). Examples of crop-related members of the family Brassicaceae include, for example, B. juncea (Indian mustard) and Thlaspi sp.

In general, the inventive technologies may be employed with any or all plants from which vegetative tissue fragments can be isolated and cultured to give whole, regenerated plants. Practically any plants can be regenerated from cultured cells or tissues, including, but not limited to, all major species of plants that produce edible sprouts. Some suitable plants include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as the sunflower etc.

Plant cells that are transformed in accordance with the present invention may be cells in culture, cells present as a disorganized mass in a callus, cells organized as leaf explants, shoot cultures, seeds, fruits, leaves, roots, or cells organized as a whole plant. Prior art teaches that, for B. juncea, explants of hypocotyls provide the greatest efficiency of transformation. However, the present invention shows that the petiole has superior shoot regeneration capacity compared with hypocotyls and cotyledons. Thus, while any of a variety of different explants (e.g., leaf explants, shoot explants, etc.) may be used in accordance with the present invention (and particularly in conjunction with inventive selection and/or regeneration media), use of petiole tissue represents a preferred embodiment of the present invention.

In accordance with the present invention, an explant of appropriate plant tissue is prepared from a plant to be transformed. Preferably, the explant is prepared from the petiole of a seedling, for example between 3 and 7 days old; most preferably the explant is prepared from a 5-day-old seedling.

The explant is prepared for transformation by a period of pre-culture. According to the present invention, this pre-culture is preferably performed on solid medium. As described below in the Example, we have found that pre-culture on agar-solidified medium can increase the frequency of shoot regeneration significantly as compared with pre-culture in liquid medium.

Preculture is preferably performed for a period approximately two days. Experimentation has shown that too little pre-culture will not trigger differentiation of explant cells and too much pre-culture may result in the overproduction of callus. A large variation in time of pre-culture can be detrimental to the susceptibility of explants to A. tumefaciens.

Bacteria

The present invention relates to transformation of plants with Agrobacterium. Agrobacterium is a representative genus of the gram-negative family Rhizobiaceae. Agrobacterium-based plasmid vectors allow the transformation of a wide range of plant species by capitalizing on the natural bacterial system for introducing DNA into the nuclear genome of plants. A. tumefaciens is a soil bacterium. It is pathogenic to a range of dicot plant species, causing the formation of crown galls or tumors at or close to infection sites. The proliferated tissue in the tumor provides the bacterium with unusual amino acids (opines), which are important to carbon and nitrogen source, at the expense of the host plant. Genes required to establish a tumor and bring about opine biosynthesis are transferred from Agrobacterium and hence this bacterium has been called Nature's genetic engineer.

Strains of Agrobacterium that are useful for Ti-vector-based plant transformation are defined by their chromosomal background and resident Ti plasmid. Significant modifications to the virulence of Agrobacterium have expanded the range of plant species that are susceptible to T-DNA transformation by improving the frequency of T-DNA transfer. Although any of the numerous strains of A. tumefaciens capable of transferring genetic material to Brassica species can be used in combination with the other variations of the present invention, particularly improved transformation, recovery, and regeneration can be achieved by using A. tumefaciens strains LBA4404, as well as other strains sharing common characteristics with these strains.

Ti-plasmids carried by A. tumefaciens may be engineered to include one or more sequences of interest to be introduced into a plant. Typically, engineered vectors will contain the sequences of interest inserted such that they are operatively linked to appropriate control sequences. For example, the bacterial genes responsible for opine expression, which are naturally carried on the Ti plasmid, can provide a convenient source of control elements to direct expression of inserted sequences. Relevant control sequences may include one or more of 1) promoter sequences; 2) 5′ untranslated sequences such as naturally occur in plants; 3) an initiation codon; 4) transcription termination sequences; 5) translation termination sequences; 6) splicing control sequences, if relevant; 7) signal sequences that direct protein translocation and/or 8) sequences that affect post-translational processing of an encoded polypeptide.

Typical engineered vectors for introducing sequences into plants also include one or more of 1) a selectable or detectable marker (if the inserted sequences are not themselves readily detectable); 2) an origin of replication; and/or 3) various other sequences necessary and/or sufficient to direct Agrobacterium-mediated transformation of plants (e.g., virulence genes, which encode a set of proteins responsible for excision, transfer, and integration of T-DNA into a plant genome; transposon sequences or other sequences that allow homologous recombination with sequences in a plant genome, etc.). Such elements may be provided on a single vector or on multiple vectors; some elements may even be provided by the plant cell being transformed. Some exemplary useful sequences and vectors are described, for example, in Lawton et al., Plant Mol. Biol. 8:315, 1987; U.S. Pat. No. 5,888,789; Fraley et al., Proc. Natl. Acad. Sci. USA 80:4803, 1983; PCT/EP99/07414, each of which is incorporated herein by reference.

According to the present invention, it is desirable to activate bacterial cells carrying an appropriate transfer vector by subculturing them in the absence of selection (e.g., in fresh medium lacking antibiotics) prior to co-culture with the plant explant. Preferably, bacteria are subcultured until they reach log growth, for example reaching an OD of about 0.4 to 0.6. Generally, bacteria are subcultured for at least about 30 minutes, more preferably at least about 60 minutes, 2 hours, 3 hours, 4 hours, 5 hours, 6 hours, 7 hours, 8 hours, or longer. In particularly preferred embodiments, bacteria are subcultured for approximately 2 hours.

Pharmaceutical Proteins

In certain preferred embodiments of the present invention, plants are transformed with sequences that encode one or more pharmaceutical proteins. Any pharmaceutical protein of interest may be expressed in plants in accordance with the present invention. Pharmaceutical proteins are proteins or polypeptides that either have pharmaceutical activity in animals, preferably in humans, or that produce, modify, or otherwise generate an agent that has pharmaceutical activity in animals, preferably humans. For instance, a protein that synthesizes or modifies a small molecule with pharmaceutical activity is a pharmaceutical protein in accordance with the present invention.

For example, particular pharmaceutical proteins of interest include, but are not limited to, hormones (insulin, thyroid hormone, catecholamines, gonadotrophines, trophic hormones, prolactin, oxytocin, dopamine, bovine somatotropin, leptins and the like), growth hormones (e.g., human growth hormone), growth factors (e.g., epidermal growth factor, nerve growth factor, insulin-like growth factor and the like), growth factor receptors, cytokines and immune system proteins (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interfersons, integrins, addressins, seletins, homing receptors, T cell receptors, immunoglobulins, soluble major histocompatibility complex antigens, immunologically active antigens such as bacterial, parasitic, or viral antigens or allergens), autoantigens, antibodies), enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (steroid binding proteins, growth hormone or growth factor binding proteins and the like), transcription and translation factors, oncoprotiens or proto-oncoprotiens (e.g., cell cycle proteins), muscle proteins (myosin or tropomyosin and the like), myeloproteins, neuroactive proteins, tumor growth suppressing proteins (angiostatin or endostatin, both which inhibit angiogenesis), anti-sepsis proteins (bacteriocidal permeability-increasing protein), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, Protein C, von Wilebrand factor, antithrombin III, glucocerebrosidase, erythropoietin granulocyte colony stimulating factor (GCSF) or modified Factor VIII, anticoagulants such as huridin) and the like.

In some embodiments of the present invention, pharmaceutical proteins are expressed in edible portions of plants, for example by transforming edible plants and/or by placing relevant genes under control of tissue-specific regulatory sequences that direct expression in edible tissues. In such embodiments, the edible portions may be harvested and formulated into a pharmaceutical, for example, to be taken orally.

Transformation

Transfer of genetic material from the bacteria to the plant is accomplished by co-culture of explant cells with bacterial cells. As discussed, the explant cells have preferably been pre-cultured in accordance with the present invention; similarly, the bacterial cells have preferably been activated in accordance with the invention.

Preferably, bacterial and plant cells are co-cultured for between about 5 minutes and 2 days, though various times are acceptable. In certain preferred embodiments, bacterial and plant cells are co-cultured for a period of about 5-30 minutes; more preferably about 20 minutes.

In particularly preferred embodiments of the invention, the co-culture is performed in the presence of a feeder layer. We have found that the presence of a feeder layer can significantly enhance the transformation rate of Brassica explants. Any appropriate cells may be used in the feeder layer; tobacco cells and/or Brassica cells are particularly preferred.

Regeneration

Transformed plant cells may be used to regenerate plants. Typically, the cells are harvested from the co-culture medium, and are characterized, for example, for viability and for the presence and/or expression of one or more transformed genes. Any available means may be employed to identify and/or characterize transformed cells. Often, as is known in the art, transformed cells are selected by growth on a selective medium. Harvested cells may be cleaned and/or dried, and may be stored under appropriate conditions of temperature, humidity, sanitation, and security to be used when necessary.

Whole plants may be regenerated from cultured protoplasts, e.g., as described in Evans et al., Handbook of Plant Cell Cultures, Vol. 1:MacMillan Publishing Co. New York, 1983); and Vasil I. R. (ed.), Cell Culture and Somatic Cell Genetics of Plants, Acad. Press, Orlando, Vol. I, 1984, and Vol. III, 1986, incorporated herein by reference.

The precise techniques used to regenerate plants from transformed protoplasts may vary from one species of plants to the next. However, those skilled in the art will appreciate that generally a suspension of transformed protoplants containing copies of the heterologous gene is first provided. Callus tissue is formed and shoots may be induced from callus and subsequently rooted. Alternatively, embryo formation can be induced from the protoplast suspension. These embryos germinate as natural embryos to form plants. Steeping the seed in water or spraying the seed with water to increase the moisture content of the seed to between 35-45% initiates germination. For germination to proceed, the seeds are typically maintained in air saturated with water under controlled temperature and airflow conditions. The culture media will generally contain various amino acids and hormones, such as auxin and cytokinins. It is also often advantageous to add glutamic acid and proline to the medium, especially for such species as alfalfa. Shoots and roots normally develop simultaneously. Efficient regeneration will depend on the medium, the genotype, and the history of the culture. If these three variables are controlled, then regeneration is fully reproducible and repeatable.

In order to obtain true-breeding transformed plants, it is common practice to self-cross the transformed plants, and to identify non-segregating, homozygous transgenic offspring. Such inbred plants produce seeds transformed with the sequence or sequences of interest; such seeds can be germinated or grown as desired. Alternatively or additionally, transformed seeds may be included in seed products and sold, for example, with instructions on how to grow the seedlings to an appropriate stage, for example allowing for a desired level or degree of expression of a transformed gene.

Inventive seeds and/or plants may also be utilized in the devevlopment of hybrids or other novel plant varieties embodying one or more desired traits (for example a transformed trait).

Administration and Pharmaceutical Compositions

Inventive transformed plants and/or the expression products of transformed sequences may be included in pharmaceutical compositions and/or administrated to human or animal subjects in need thereof. Preferred recipient subjects include vertebrates, preferably mammals, more preferably humans. Preferred non-human subjects include, for example, bovines, ovines, canines, felines, etc.

Transformed plants themselves may be formulated into pharmaceutical compositions according to the present invention. Alternatively, expression products (or products of expression products) of transformed sequences may be isolated and/or purified from plant tissues, and may then be formulation into pharmaceutical compositions.

Inventive pharmaceutical compositions may be formulated for any mode of delivery including, for example, oral, enteral, nasal, parenteral, intramuscular or intravenous, rectal, vaginal, topical, ocular, pulmonary, or by contact application. In certain preferred embodiments of the invention, the pharmaceutical compositions are formulated for oral administration. For such embodiments, it may often be desirable to utilize edible plants, or at least plants with edible portions, preferably in which transformed sequences are expressed.

The pharmaceutical compositions of the present invention typically include an effective amount of a transformed plant or expression product together with one or more organic or inorganic, liquid or solid, pharmaceutically suitable carrier materials. The compositions may be prepared in dosage forms such as tablets, capsules, troches, dispersions, suspensions, solutions, capsules, creams, ointments, aerosols, powder packets, liquid solutions, solvents, diluents, surface active agents, isotonic agents, thickening or emulsifying agents, preservatives, solid bindings as long as the relevant biological activity is not destroyed by the dosage form.

Pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator (see also Remington 's Pharmaceutical Sciences, Fifteenth Edition, E. W. martin (Mack Publishing Co., Easton Pa., 1975). For example, the protein may be provided as a pharmaceutical composition by means of conventional mixing granulating dragee-making, dissolving, lyophilizing, or similar processes.

Where an isolated protein or other (e.g., small molecule agent) is employed as an active ingredient in a pharmaceutical composition in accordance with the present invention, it may be desirable to prolong the effect of a pharmaceutical preparation by slowing the absorption of the relevant active ingredient. This may be accomplished, for example, by the use of a liquid suspension of crystalline or amorphous material with poor water solubility. The rate of absorption of the active ingredient then depends upon its rate of dissolution, which in turn, may depend upon size and form. Alternatively, delayed absorption of an active ingredient may be accomplished by dissolving or suspending the active ingredient in an oil vehicle (e.g., to generate a depot). Injectable depot forms are made by forming microencapsule matrices of the active ingredient in biodegradable polymers such as polylactide-polyglycolide. Depending upon the ratio of active ingredient to polymer, and the nature of the particular polymer employed, the rate of release can be controlled. Examples of other biodegradable polymers include poly(orthoesters) and poly(anhydrides). Depot injectable formulations may also prepared by entrapping the active ingredient in liposomes or microemulsions, which are compatible with body tissues.

Internally-administered pharmaceutical compositions may be introduced in solid, semi-solid, suspension or emulsion form and may be compounded with any pharmaceutically acceptable carriers, such as water, suspending agents, and emulsifying agents. Inventive active ingredients may also be administered by means of pumps or sustained-release forms, especially when administered as a preventive measure, so as to prevent the development of disease in a subject or to ameliorate or delay an already established disease.

The plants or pharmaceutically active proteins produced according to the present invention are particularly well suited for oral administration as pharmaceutical compositions. For example, harvested seedlings may be processed in a variety of ways, e.g., air drying, freeze drying, extraction etc., depending on the properties of the desired pharmaceutical product. In preferred embodiments, such compositions as described above are ingested orally alone or ingested together with food or feed or a beverage. Compositions for oral administration include plants; extractions of the plants; and proteins or other agents purified from plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze dried, air dried, or spray dried. For example, the plants may be air dried by placing them in a commercial air dryer at about 120 degrees Fahrenheit until the biomass contains less than 5% moisture by weight. The dried plants are stored for further processing as bulk solids or further processed by grinding to a desired mesh sized powder. Alternatively, freeze-drying may be used for products that are sensitive to air-drying. Products may be freeze dried by placing them into a vacuum drier and dried frozen under a vacuum until the biomass contains less than about 5% moisture by weight. The dried material can be further processed as described herein.

Herbal preparations are well known in the art. Herbal preparations that may be used to administer the plants of the present invention include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

Those skilled in the art will appreciate that a particularly preferred method of obtaining a desired pharmaceutically active protein or other agent (e.g., small molecule) is by extraction. Fresh plants may be extracted to remove the desired products from the residual biomass, thereby increasing the concentration and purity of the products. Plants may also be extracted in a buffered solution. For example, the fresh harvested plants may be transferred into an amount of ice-cold water at a ratio of one to one by weight that has been buffered with, e.g., phosphate buffer. Protease inhibitors can also be added as required. The plants can be disrupted by vigorous blending or grinding while suspended in the buffer solution and the extracted biomass removed by filtration or centrifugation. The protein product carried in solution can be further purified by additional steps or converted to a dry powder by freeze-drying or precipitation. Extraction can also be carried out by pressing. Live plants can also be extracted by pressing in a press or by being crushed as they are passed through closely spaced rollers. The fluids expressed from the crushed plants are collected and processed according to methods well known in the art. Extraction by pressing allows the release of the products in a more concentrated form. However, the overall yield of the product may be lower than if the product were extracted in solution.

The plants, extractions, powders, dried preparations and purified products, etc., can also be in encapsulated form with or without one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active pharmaceutical protein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In other particularly preferred embodiments, a transgenic plant expressing a pharmaceutically active protein of the present invention, or biomass of transgenic plants, is administered orally as medicinal food. Such edible compositions are consumed by eating raw, if in a solid form, or by drinking, if in liquid form. In a preferred embodiment, the transgenic plant material is directly ingested without a prior processing step or after minimal culinary preparation. For example, the pharmaceutically active protein is expressed in a plant of which can be eaten directly. For example, the protein is expressed in alfalfa, mung bean, or spinach or lettuce leaf, etc. In an alternative embodiment, the plant biomass is processed and the material recovered after the processing step is ingested.

Processing methods preferably used in the present invention are methods commonly used in the food or feed industry. The final products of such methods desirably include a substantial amount of an expressed pharmaceutically active protein (or product thereof), and are preferably conveniently eaten or drunk. The final product may also be mixed with other food or feed forms, such as salts, carriers, favor enhancers, antibiotics, and the like, and consumed in solid, semi-solid, suspension, emulsion, or liquid form. In another preferred embodiment, such methods include a conservation step, such as, e.g., pasteurization, cooking, or addition of conservation and preservation agents. Any plant is used and processed in the present invention to produce edible or drinkable plant matter. The amount of pharmaceutically active protein (or product) in an edible or drinkable plant preparation may be tested by methods standard in the art, e.g., gel electrophoresis, Elisa, or Western blot analysis, using an antibody specific for the protein. This determination may be used to standardize the amount of protein ingested. For example, the amount of therapeutically active protein in a plant juice determined and regulated, for example, by mixing batches of product having different levels of protein so that the quantity of juice to be drunk to ingest a single dose can be standardized. The contained, regulatable environment of the present invention, however, should minimize the need to carry out such standardization procedures.

A pharmaceutically active protein produced in a plant and eaten by a host is absorbed by the digestive system. One advantage of the ingestion of a plant or plant preparation, particularly intact plant or plant biomass that has been only minimally processed, is to provide encapsulation or sequestration of the protein in cells of the plant. Thus, the protein may receive at least some protection from digestion in the upper digestive tract before reaching the gut or intestine and a higher proportion of active would be available for uptake.

The pharmaceutical compositions of the present invention can be administered therapeutically or prophylactically. In certain preferred embodiments, the compositions may be used to treat or prevent a disease. For example, any individual who suffers from a disease or who is at risk of developing a disease may be treated. It will be appreciated that an individual can be considered at risk for developing a disease without having been diagnosed with any symptoms of the disease. For example, if the individual has a particular genetic marker identified as being associated with increased risk for developing a particular disease, that individual will be considered at risk for developing the disease. Similarly, if members of an individual's family have been diagnosed with a particular disease, e.g., cancer, the individual may be considered to be at risk for developing that disease.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories which can be prepared by mixing the compositions of this invention with suitable non-irritating excipients or carriers such as cocoa butter, polyethylene glycol or a suppository wax which are solid at ambient temperature but liquid at body temperature and therefore melt in the rectum or vaginal cavity.

Dosage forms for topical or transdermal administration of a pharmaceutical composition of this invention include ointments, pastes, creams, lotions, gels, powders, solutions, sprays, inhalants or patches. The transformed plant or product thereof, is admixed under sterile conditions with a pharmaceutically acceptable carrier and any needed preservatives or buffers as may be required. Ophthalmic formulation, eardrops, and eye drops are also contemplated as being within the scope of this invention.

Additionally, the present invention contemplates the use of transdermal patches, which have the added advantage of providing controlled delivery of a pharmaceutically active protein or other agent to the body. Such dosage forms can be made by suspending or dispensing the pharmaceutically active protein or agent in the proper medium. Absorption enhancers can also be used to increase the flux of the pharmaceutically active protein or agent across the skin. The rate can be controlled by either providing a rate controlling membrane or by dispersing the pharmaceutically active protein or agent in a polymer matrix or gel.

Inventive pharmaceutical compositions are administered in such amounts and for such time as is necessary to achieve the desired result. As described above, in certain embodiments of the present invention a “therapeutically effective amount” of a pharmaceutical composition is that amount effective for treating, attenuating, or preventing a disease in a host. Thus, the “amount effective to treat, attenuate, or prevent disease”, as used herein, refers to a nontoxic but sufficient amount of the pharmaceutical composition to treat, attenuate, or prevent disease in any host. As but one example, the “therapeutically effective amount” can be an amount to treat, attenuate, or prevent diabetes.

The exact amount required may vary from subject to subject, depending on the species, age, and general condition of the subject, the stage of the disease, the particular pharmaceutical mixture, its mode of administration, and the like. The plants of the invention and/or protein or other preparations thereof are preferably formulated in dosage unit form for ease of administration and uniformity of dosage. The expression “dosage unit form,” as used herein, refers to a physically discrete unit of pharmaceutically active protein appropriate for the patient to be treated. It will be understood, however, that the total daily usage of the compositions of the present invention are preferably decided by an attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex of the patient, diet of the patient, pharmacokinetical condition of the patient, the time of administration, route of administration, and rate of excretion of the specific compound employed; the duration of the treatment; drugs used in combination or coincidental with the specific compound employed; and like factors well known in the medical arts.

It will also be appreciated that the pharmaceutical compositions of the present invention can be employed in combination therapies, that is, the pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-cancer agent), or they may achieve different effects.

The Brassica transformation and regeneration system described above has been found to be rapid and efficient. A sufficient percentage of the co-cultivated explants are transformed in order to provide an economic system for transforming Brassica.

The following Example is offered by way of illustration and not by way of limitation.

### Exemplification

Materials and Methods

Construction of Transformation Vector:

Entechon GmbH (Germany) was contracted to synthesize the gene for Human Growth Hormone (HGH) with optimized codon usage for Brassicaceae. Two binary transformation vectors were constructed carrying the HGH gene under transcriptional control of either the Cauliflower Mosaic Virus 35S promoter or the promoter from the 18.2 kDa heat shock protein gene (HSP 18.2) of Arabidopsis thaliana (Matsuhara et al., Plant J. 22(1):79, 2000) The constructs driven by the 35S promoter also contained the omega translational enhancer sequence (Ω) from tobacco mosaic virus (Gallie, Nucleic Acids Res. 30(15):3401, 2002). In addition to the HGH expression cassette, both vectors were constructed with a cassette for selection of kanamycin resistant plant cells consisting of the NPTII gene under control of the Agrobacterium tumefaciens nopaline synthase (NOS) promoter and transcriptional terminator, and a cassette for visual screening of transformed plants consisting of the β-glucuronidase (GUS) gene under control of either the HSP18.2 promoter or the 35S promoter/Ω enhancer and NOS terminator. In each vector the promoter of the GUS expression cassette was the same as the HGH cassette. All plasmid constructs were confirmed by restriction mapping, polymerase chain reactions (PCR), and DNA sequencing. Two expression vectors were transformed into A. tumefaciens strain LBA4404 (Life Technologies, Gaithersburg, Md., catalog #18313-015) by electroporation. The transformed strains were confirmed by single colony PCR using oligonucleotide primers specific for HGH and GUS (see section on PCR analysis).

Plant Material and Tissue Culture:

Seeds of Brassica juncea (accession no. 426308) were surface-sterilized with 70% (v/v) ethanol for 2 min, then 35% (v/v) commercial Clorox plus 0.1% (v/v) Tween20 for 15 min with vigorous shaking, followed by 4 rinses in sterile distilled water. They were then germinated on half-strength hormone-free Murashige Skoog (MS) medium (1962) (Life Technologies, Gaithersburg, Md.) solidified with 2% (w/v) agar in Magenta Vessels (Sigma V8505), with about 60 seeds in each vessel. Plants were grown for 2 days in the dark and 3 days in light at 25° C., until the cotyledons were fully expanded and hypocotyls were 3 to 4 cm long. Segments of cotyledonary petiole without shoot apex were dissected quickly on water soaked, sterile filter paper. They were then cultured on pre-infection medium under a 16:8 hour (light:dark) photoperiod and at 25° C.

Preparation of the Feeder Layer:

A suspension culture of tobacco was prepared as follows. Seeds of tobacco (Samsun, wild type) were surface-sterilized with 70% (v/v) ethanol for 2 min, then with 25% (v/v) commercial Clorox and 0.1% (v/v) Tween20 for 10 min with vigorous shaking, followed by 5 rinses in sterile distilled water. They were then germinated on full-strength hormone-free Murashige Skoog's (MS) medium (1962) in Magenta Vessels (Sigma V8505). Seedlings were grown for 10 to 15 days in continuous light at 25° C., until they were about 1 to 2 inches tall. Leaf tissue was quickly dissected into 1-cm2 segments on wet sterile filter paper. They were then cultured on callus-inducing medium composed of MS salts with vitamins (Sigma, catalog # M5519), 3% (w/v) sucrose, 2.5 g L⁻¹ Gelrite, 5 mM 6-benzylaminopurine (BAP), and 2.5 mM naphthalene acetic acid (NAA) (the complete medium adjusted to pH 5.8) under a 16:8 hour (light:dark) photoperiod at 25° C. After about 2 weeks, light green clusters of proliferating cells (calli) were transferred to 250 mL Erlenmeyer flasks containing liquid suspension medium composed of MS salts and vitamins, 3% (w/v) sucrose and 1 mg L⁻¹ 2,4-dichlorophenoxyacetic acid (2,4-D) (medium adjusted to pH 5.8). The cultures were grown under continuous light at 25° C. with shaking at 100 rpm. Cell suspensions were established by several subcultures during which large cell clumps were removed. One day prior to use, 1.5 mL of 5 to 6 day old tobacco suspension cells were plated onto pre-infection medium and a sterile filter paper was placed on their surface.

Transformation:

Pre-culture:

Explants prepared as described above were pre-cultured for 2 days on solid pre-infection medium containing MS salts with vitamins (Sigma, catalog #M5519), 3% (w/v) sucrose, 2.5 g L⁻¹ Gelrite, 2 mg L⁻¹6-benzylaminopurine (BAP), 0.1 mg L⁻¹ naphthalene acetic acid (NAA) under a 16:8 hour (light:dark) photoperiod and 25° C.

Bacterial Activation:

A. tumefaciens strain LBA4404 carrying the transformation plasmids were grown for 2 days in 50 mL liquid YEP medium containing 1 g L⁻¹ yeast extract, 5 g L⁻¹ beef extract, 5 g L⁻¹ bacto-peptone, 5 g L⁻¹ sucrose, 0.5 g L⁻¹ MgSO₄.7H₂O and 100 mg L⁻¹ kanamycin in a 28° C. shaker at 190 rpm. The density of bacteria was adjusted to A₆₀₀=0.4 with fresh YEP medium without antibiotic, then cultured about 2 more hours until the A₆₀₀ was 0.6. The subculture and growth is referred to in the text as the “activation treatment”. The bacteria were harvested by centrifugation at 4800-rpm for 15 min at 4° C. and then bacterial pellet resuspended in MS liquid medium.

Infection:

After 2 days on pre-infection medium, the explants were transferred into the A. tumefaciens suspension and incubated for 20 min. Afterwards they were blotted dry with sterile filter paper and transferred onto the filter paper atop the freshly prepared feeder layer. After 2 days the explants were rinsed in medium containing MS salt, 3% (w/v) sucrose, and 500 mg L⁻¹ carbencillin for 40 min on a shaker at 100 rpm. The explants were then transferred to the selective media.

Selection:

Six different selection media were tested containing different hormone combinations. These were, S-1 (0.22 mg L⁻¹ thidiazuron [TDZ], and 2 mg L⁻¹ 2-isopentenyladenine [2iP]), S-2 (2 mg L⁻¹ TDZ+0.1 mg L⁻¹ indole-3-acetic acid [IAA]); S-3 (2 mg L⁻¹ Zeatin), S-4 (2 mg L⁻¹ BAP, 0.1 mg L⁻¹ napthalene acetic acid [NAA]), S-5 (2 mg L⁻¹ TDZ, 2 mg L⁻¹ benzylaminopurine [BAP], and 0.1 mg L⁻¹ NAA), S-6 (2 mg L⁻¹ TDZ and 0.1 mg L⁻¹ NAA). Other components of selective medium included 40 mg L⁻¹ kanamycin, 500 mg L⁻¹ carbencillin, and 30 μM silver nitrate (AgNO₃). Kanamycin resistant calli and shoots produced from explants were counted to evaluate the rate of regeneration. Kanamycin resistant calli and shoots were also checked for transformation by histochemical detection of GUS reporter gene activity. The shoots were transferred onto rooting medium in culture jars (Sigma, V8630). Rooting medium contained MS salts, 3% (w/v) sucrose, 2.5 g L⁻¹ Gelrite, 2 mg L⁻¹ indole-3-butyric acid (IBA), and 500 mg L⁻¹ carbencillin.

Histochemical Assay for β-glucuronidase (GUS):

Tissue sections or organs of plants were incubated overnight at 37° C. in a solution of X-Gluc consisting of 10 mM ethlenediaminetetraacetic acid, 100 mM sodium phosphate (pH 7.0), 0.5 mM potassium ferricyanide, 0.5 mM potassium ferrocyanide, and 0.1% (w/v) X-gluc (Sigma B4527). Chlorophyll was removed from the tissues by washing in 70% (v/v) ethanol for 15 min, 90% (v/v) ethanol for 15 min, and then 100% (v/v) for 1 hour. The sections were examined for blue color formation using a microscope.

PCR Analysis:

To detect the presence of the HGH gene and the GUS gene two pairs of primers were designed (forward HGH primer: 5′ ATG GCA ACT GGA TCA AGG ACT 3′, reverse HGH primer: 5′ TCA ACT TTA TCC ATA TCC TTC CTA 3′; forward GUS primer: 5′ CGT CCT GTA GAA AC 3′, reverse GUS primer: 5′ TGA TGA TAA TCG GCT GAT GC 3′). Genomic DNA extraction and PCR were carried out using the REDExtract-N-Amp™ Plant PCR Kits (Sigma) according to the instructions.

Results

Regeneration of Kanamycin-Resistant Plantlets after Transformation:

Around the 3^{th} to 4^{th} day after transformation, many roots started to grow from the cut regions of the explants on the selective medium. Some white or light green calli and purple or green protuberances of shoot became visible on the 5^{th} to 6^{th} day. By the 10^{th} to 15^{th} day the shoots had grown to around 1 to 1.5 cm in length with 3 to 5 leaves. Most shoots emerged from calli, not directly from explants. Generally, 1 to 7 shoots would be produced from one cut end of an explant. The green and healthy shoots were removed to the rooting medium, selecting only a single shoot from each cut surface. During this process most of the purple shoots gradually became white. Roots were not produced from white shoots and they ultimately died in culture. However, many of them were positive for GUS expression, therefore they are likely to be transformed even though they are unable to survive on selection medium. The majority of transgenic plants that were transferred to soil had normal morphology, and were able to flower and set seed. A few of them had unusual morphologies such as stunted growth, yellow leaves, and sterility.

Comparison of the Effect of Cultural Conditions on Shoot Regeneration Frequency:

Several different parameters were examined with regard to promotion of transformation and shoot regeneration. Three different seedling tissues were compared as was pre-culture method, activation treatment of A. tumefaciens before infection, feeder layer treatment, washing of explants after co-culture with A. tumefaciens, and 6 different selective media (S-1 to S-6). The results were as follows:

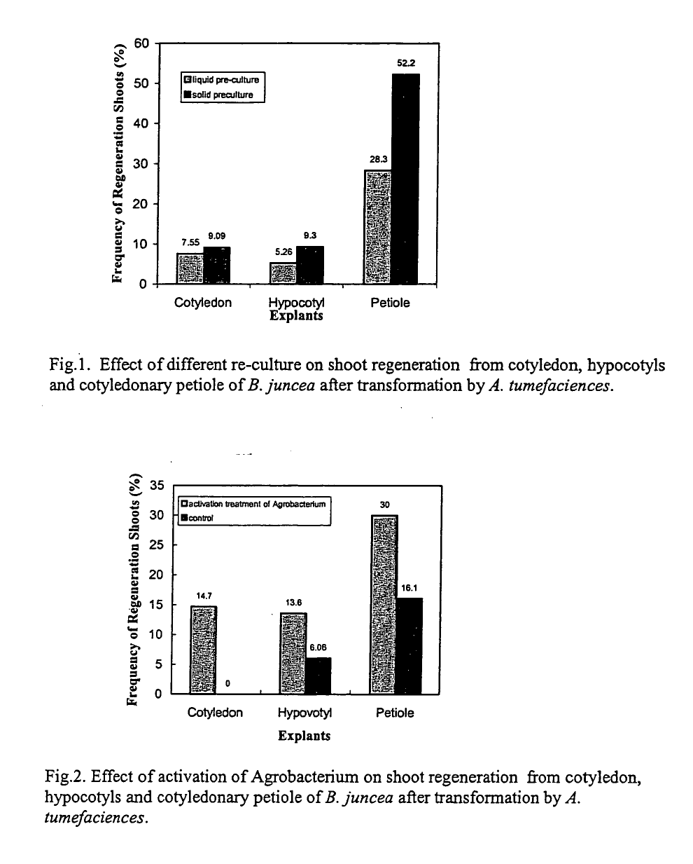

a. Pre-culture on agar solidified medium produced 52.2% frequency of shoot regeneration from petiole explants compared with 28.3% when pre-cultured on liquid medium (FIG. 1). The experiment also showed that the petiole has superior shoot regeneration capacity compared with hypocotyls and cotyledons. Previous reports on transformation of B. juncea used only hypocotyls. Pre-culturing in shaking liquid medium caused explants to grow quickly compared with culture on agar-solidified medium. The early fast growth of explants generally resulted in poor shoot regeneration.

b. Activation treatment of A. tumefaciens cells increased the capacity for shoot regeneration from all the tissues tested (FIG. 2). Presumably, the activation treatment increased the infectivity of the A. tumefaciens.

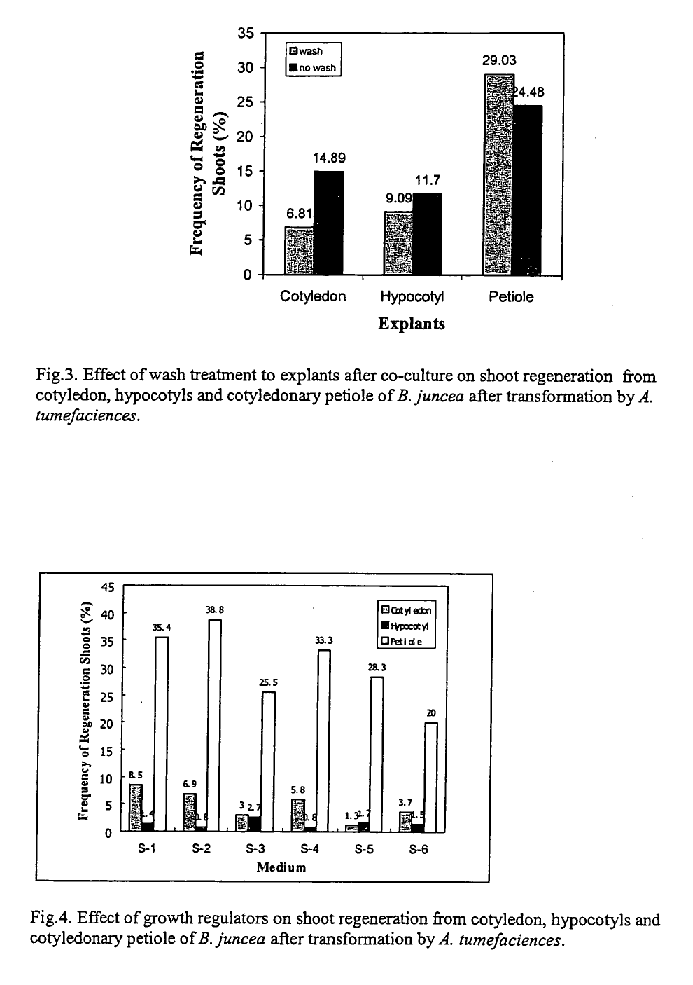

c. No significant difference was found between washing and not washing explants after co-culture with A. tumefaciens (FIG. 3).

d. Of the six media with different hormone combinations S-2 (2 mg L⁻¹ TDZ+0.1 mg L⁻¹ IAA) was found to be superior (FIG. 4). On this medium kanamycin resistant shoots were formed at a frequency of 38.8%.

In summary, the results show that explant source, pre-culture treatment, and feeder layer treatment are affect the efficiency of shoot regeneration after transformation with A. tumefaciens. Shoot regeneration was extremely inefficient in the absence of pre-culture and feeder layer treatment (approximately 3% of explants). The time of culture on the feeder layer was also found to be important in that a 2-day treatment was better than a 4-day treatment. It appeared that 4-day treatment on the feeder layer produce very rapid growth of callus, which inhibited shoot regeneration.

Optimized protocol to Achieve Superior High Transformation Efficiency of B. juncea:

The results indicate that a variety of improvements can be made to achieve superior, high transformation efficiency of B. juncea. These improvements can be summarized as follows:

1. Five-day-old seedlings are preferably used to obtain explants.

2. Two day pre-culture on solid pre-culture medium is highly beneficial. One day and 3 days pre-culture do not trigger dedifferentiation of explant cells or overproduction of callus. Both of are detrimental to the susceptibility of explants to A. tumefaciens.

3. Activation of A. tumefaciens by subculture for two hours on fresh medium lacking antibiotics is important to effect efficient infection of explants.

4. The use 4 to 5 day old tobacco suspension cells as a feeder layer during the period when explants are co-cultured with A. tumefaciens is also desirable. Without wishing to be bound by any particular theory, we propose that diffusible factors produced by the feeder-layer are important in induction of shoot formation, or have a protective function against the overgrowth of A. tumefaciens cells.

5. The use of a combination of TDZ (2 mg L⁻¹ ) and IAA (0.1 mg L⁻¹ ) are superior to the production and growth of kanamycin-resistant shoots. Frequent subculture of shoots on with fresh medium is beneficial.

6. After antibiotic-resistant shoots are regenerated, the use of 2 mg L⁻¹ IBA provides superior ability of shoots to form roots.

Identification of Transgenic Plants of B. juncea Under Optimizing Conditions:

Under the above optimized conditions, a plasmid containing the HGH gene driven by the 35S promoter/Ω translation enhancer and one driven by the HSP18.2 promoter were used to transform B. juncea. For the 35S promoter/Ω translation enhancer construct 62 independent, kanamycin-resistant lines were selected and 32 of these were screened by PCR revealing 30 PCR-positive lines. The overall frequency of transformation for this construct was calculated as 58% (Table 1). For the HSP18.2 promoter construct 12 independent, kanamycin-resistant lines were selected and 10 of these were screened by PCR revealing 7 PCR-positive lines. The overall frequency of transformation for this construct was calculated as 22% (Table 1). The lines are currently being examined for expression of HGH.

According to the present invention, a superior, high efficiency of transformation of B. juncea was obtained in this study (58% of initial explants for an HGH expression construct). The transgenic plants could easily be obtained in one month. In previous publications, the efficiency of transformation of B. juncea was reported as 3 to 9 percent, and required 6 months. There are several factors that contribute to the high efficiency reported here.

Most previous researchers used cotyledon and hypocotyls as explants. The present invention that the petiole is a superior explant source. The efficiency of shoot regeneration from petioles is about 48 fold greater than from hypocotyls, and 6 fold greater than cotyledons under the same treatment and culture conditions.

It is the wounded cut end of tissue explants that produce de-differentiated callus cells that are competent for transformation by A. tumefaciens. Our experiments have indicated that the preculture conditions contribute to the development of transformation competency.

The feeder layer is another important factor that increases transformation efficiency. We propose that diffusible substances, perhaps hormone or other signaling molecules, are transferred from the feeder cells that positively influences the interaction between explants and A. tumefaciens (Jeroen et al., Plant Cell Report 12:644, 1993; McCormick et al., Plant Cell Report 5:81, 1986). Our experiments have shown that feeder layer treatment is important to the high efficiency of transformation on B. juncea.

Silver nitrate is an inhibitor of ethylene production. Ethylene is a plant hormone controlling shoot organogenesis of B. campestris (Chi et al., Plant Physiol. 96(1):178, 1991). The presence of silver nitrate in the selective medium greatly enhanced shoot regeneration from several recalcitrant genotypes of B. juncea and B. campestris (Chi et al., Plant Sci. 64:243, 1989; Chi et al., Plant Cell Rep. 9:195, 1990), B. napus and B. oleracea (De Block et al., Plant Physiol. 96(1):178, 1989).

TDZ has proven to be very effective in inducing shoot regeneration in vitro of kiwi (Suezawa et al., Sci. Hort. 37:123, 1988), apple ( Fasolo et al., Plant Cell Tissue Organ Cult. 16:75, 1989), grape ( Matsuta et al., Plant Cell Rep. 7:684, 1989), pear (Leblay et al., Plant Cell Tissue Organ Cult.25:99, 1991), pea (Bohmer et al., Plant Cell Rep. 10:26, 1995), peanut ( Kanyand et al., Plant Cell Rep. 14:1, 1994), eggplant (Magioli et al., Plant Cell Rep. 17:661, 1998), castor bean (Sujatha et al., Plant Cell Rep. 17:561, 1998), cherry (Hammatt et al., Plant Cell Rep. 17:526, 1998), legumes (Collen et al., Plant Cell Rep. 19:13, 1999), and other some woody species (Huetteman et al., Plant Cell Organ Tissue Cult. 33:105, 1993). In this study TDZ was tested for the first time in the transformation of B. juncea. Our experiment showed that TDZ combined with IAA greatly shorten the time to shoot formation. For example, on TDZ/IAA medium shoots grew to 1 cm in length with 3 to 4 leaves seven days after transformation, compared with 18 to 20 days on medium with NAA and BAP. TDZ (thidiazuron) is a substituted phenylurea that is commercially used as a defoliant for cotton. It also has potent cytokinin-like activity (Wang et al., Phytochemistry 25:311, 1986; Fiola et al., Plant Cell Tissue Organ Cult. 20:223, 1990; Saxena et al., Planta 187:421, 1992).

The gene transfer system described in this study provided a simple and rapid method to obtain super high transformation efficiency of B. juncea by A. tumefaciens based transformation . The method provides great potential for the genetic studies of B. juncea and its use in applied methods to produce agronomically important traits and production of therapeutic products in plants.

TABLE 1
Regeneration and identification of transgenic shoots and plants from
petioles of B. juncea infected with A. tumefaciens LBA4404.

| | Number of | Number of | Frequency of | Number of | Number of | Frequency of | Lines Screened | Lines Shown to |
|---|---|---|---|---|---|---|---|---|
| | Explants | Kanamycin | Isolating | Isolated Shoots On | Kanamycin | Root Formation | by PCR for the | Contain the |
| 35S- | 237 | 168 | 71% | 69 | 62 | 87% | 32 | 30 |
| HGH | | | | | | | | |
| HSP- | 38 | 12 | 32% | 12 | 12 | 100% | 10 | 7 |
| HGH | | | | | | | | |

| | | | Number of Lines |
|---|---|---|---|
| | Frequency of | Overall | Showing Expression of |
| | Recovery of Lines | Frequency of | Recombinant Protein by |
| 35S- | 94% | 58% | ? |
| HGH | | | |
| HSP- | 70% | 22% | ? |
| HGH | | | |

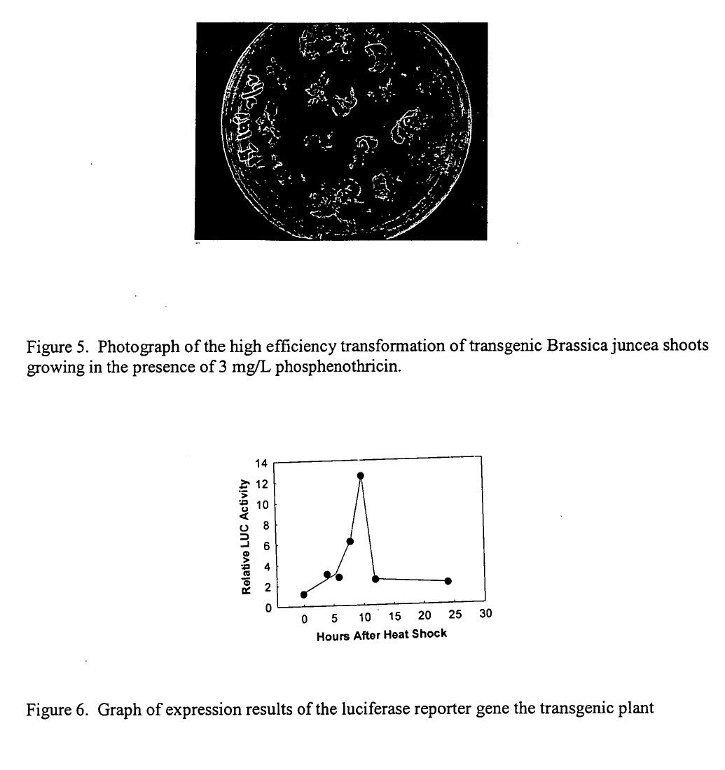

In other embodiments, the BAR gene which encodes for resistance to the herbicide phosphenothricin was used for the regeneration method of the present invention. The selection protocol was optimized using 3 mg/L phosphenothricin. 96.79% transformation efficiency was achieved using the method of the present invention. Of 468 explants, 453 produced one to multiple transgenic shoots. FIG. 5 shows transgenic Brassica juncea shoots growing in the presence of 3 mg/L phosphenothricin. The vector used for transformation carried an expression cassette in which the Firefly luciferase gene is controlled by the Arabidopsis thaliana HSP18.2 heat shock promoter. The expression of the luciferase reporter gene was studied in one of the transgenic plants. FIG. 6 displays the results of the luciferase activity in plants exposed to heat shock. Explants were exposed for 2 hours to 37° C. under 100% humidity conditions. The explants were then transferred to 24° C. and luciferase activity was measured over a period of 24 hours. Luciferase activity could not be detected in plants that were not exposed to heat shock.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method for improved transformation efficiency of a plant, comprising:
providing a plant cell;
pre-culturing said plant cell;
providing a feeder layer;
activating a bacteria;
contacting the cultured plant cell and bacteria in the presence of the feeder layer to cause transformation of the plant cell;
identifying a transformed plant cell; and
allowing the transformed cell to grow into a plant.

2. The method of claim 1, wherein the plant cell is a member of the family Brassicaceae.

3. The method of claim 2, wherein the plant cell is a member of the genus Brassica.

4. The method of claim 3, wherein the plant cell is a member of the species Brassica juncea.

5. The method of claim 1, wherein the plant cell is extracted from a seedling approximately five days old.

6. The method of claim 1, wherein the plant cell is pre-cultured on a solid pre-infection medium.

7. The method of claim 6, wherein the plant cell is pre-cultured on an agar solidified medium.

8. The method of claim 6, wherein the plant cell is pre-cultured for approximately two days.

9. The method of claim 1, wherein the plant cells are pre-cultured in liquid medium.

10. The method of claim 1, wherein the bacteria is a member of the genus Agrobacterium.

11. The method of claim 10, wherein the bacteria is a member of the species Agrobacterium tumefaciens.

12. The method of claim 11, wherein the bacteria strain is LBA4404.

13. The method of claim 1, wherein the bacteria is activated by subculture on a fresh medium.

14. The method of claim 13, wherein the bacteria is activated for approximately two hours.

15. The method of claim 13, wherein the fresh medium lacks antibiotics.

16. The method of claim 1, wherein the feeder layer comprises tobacco cells.

17. The method of claim 16, wherein the tobacco cells are approximately four days old.

18. The method of claim 16, wherein the tobacco cells are approximately five days old.

19. The method of claim 1, wherein the plant cells are washed after contacting the plant cells with the bacteria.

20. The method of claim 1, wherein the plant cells are not washed after contacting the plant cells with the bacteria.

21. The method of claim 1, wherein the selecting of transformed cells uses different selection media containing different hormone combinations selected from group consisting of S-1 (0.22 mg L⁻¹ thidiazuron [TDZ], and 2 mg L⁻¹ 2-isopentenyladenine [2iP]), S-2 (2 mg L⁻¹ TDZ+0.1 mg L⁻¹ indole-3-acetic acid [IAA]); S-3 (2 mg L⁻¹ Zeatin), S-4 (2 mg L⁻¹ BAP, 0.1 mg L⁻¹ napthalene acetic acid [NAA]), S-5 (2 mg L⁻¹ TDZ, 2 mg L⁻¹ benzylaminopurine [BAP], and 0.1 mg L⁻¹ NAA); and S-6 (2 mg L⁻¹ TDZ and 0.1 mg L⁻¹ NAA).

22. The method of claim 21, wherein the selection media is S-2 (2 mg L⁻¹ TDZ+0.1 mg L⁻¹ indole-3-acetic acid [IAA]).

23. The method of any one of the prior claims, wherein the plant cell becomes transformed with a gene encoding a pharmaceutical protein.

24. The method of claim 23 wherein the plant includes edible portions.

25. The method of claim 24 wherein the pharmaceutical protein is expressed in the edible portions of the plant.

26. The method of claim 25, further comprising a step of harvesting the edible portions.

27. The method of claim 26, further comprising a step of formulating the harvested edible portions into a pharmaceutical composition.

28. The method of claim 23, further comprising a step of formulating the pharmaceutically active protein into a pharmaceutical composition.

29. The method of claim 28, wherein the step of formulating comprises isolating the pharmaceutically active protein away from plant tissue.

30. The method of claim 23, wherein the pharmaceutically active protein activates a non-protein pharmaceutically active agent.

31. The method of claim 30, further comprising a step of formulating the non-protein pharmaceutically active agent into a pharmaceutical composition.

32. The method of claim 31, wherein the step of formulating comprises isolating the pharmaceutically active agent away from plant tissue.

33. A method of transforming B. juncea cells by contacting the cells with A. tumefaciens cells carrying nucleic acid sequences to be transferred so that at least about 35% of the B. juncea cells are transformed with the nucleic acid sequences.

34. The method of claim 33, wherein at least about 40% of the B. juncea cells are transformed with the nucleic acid sequences.

35. The method of claim 33, wherein at least about 45% of the B. juncea cells are transformed with the nucleic acid sequences.

36. The method of claim 33, wherein at least about 50% of the B. juncea cells are transformed with the nucleic acid sequences.

37. The method of claim 33, wherein at least about 55% of the B. juncea cells are transformed with the nucleic acid sequences.

38. The method of claim 33, wherein at least about 58% of the B. juncea cells are transformed with the nucleic acid sequences.

39. A method of transforming B. juncea cells with a desired nucleic acid sequence by contacting the cells with A. tumefaciens so that B. juncea plants transformed with the desired sequence are produced within about 3 months.

40. The method of claim 39 wherein the plants are produced within about 2 months.

41. The method of claim 39 wherein the plants are produced within about 1 month.
